(19) United States
(12) Patent Application Publication
(10) Pub. No.: US 2011/0196469 A1
(43) Pub. Date: Aug. 11, 2011

(54) Navigation Guide Wire Through an Anatomical Structure Having Branched Ducts

(75) Inventor: Alessandro Lualdi, Marcallo con Casone (Milano) (IT)

(73) Assignee: EVR MEDICAL S.AR.L., Luxembourg (LU)

(21) Appl. No.: 13/062,116

(22) PCT Filed: May 18, 2009
(86) PCT No.: PCT/IB2009/052064

§ 371 Date: Apr. 28, 2011

(30) Foreign Application Priority Data
Sep. 5, 2008 (IT) MI2008A001589

(51) Int. Cl. A61F 2/84

(52) U.S. Cl. 623/1.11

(57) ABSTRACT

A navigation guide wire has a proximal portion with a first flexural rigidity, a distal portion with a second flexural rigidity greater than or equal to the first flexural rigidity, and an intermediate potion with a third flexural rigidity substantially less that both said first and second flexural rigidities. This allows a considerable direction change between said proximal and distal portions.

### FIELD OF THE INVENTION

This invention generally relates to navigation guide wires through an anatomical structure having branched ducts, such as, for example, vascular ducts.

### BACKGROUND OF THE INVENTION

Medical devices such as, for example, endovascular or intravascular devices, have been used for many years, for example, in order to perform medical interventions. A medical device such as, for example, an intravascular device, can be introduced in a patient's anatomical structure, for example, in vascular ducts, in an easily accessible length of these ducts, and then guided through the patient's anatomical structure to the desired position. In order to observe the position of the medical device, for example, the distal end thereof, X-ray devices are employed which allow the operator to detect the medical device position and, in case of an injection of a contrast fluid into the ducts, to characterize the two-dimensional or three-dimensional appearance of the patient's anatomical structure, thus allowing to guide the device and negotiate branches through ducts bifurcations, thereby reaching the desired position.

Such medical devices, commonly known as guide wires, or GWs, require sufficiently solid structures in order to allow transmitting the thrust exerted by the operator to the proximal end, which is located outside the patient's body, with a substantial quickness or promptness to the distal portions.

At the same time, these medical devices need to have a sufficient flexibility, which allows the guide wire to follow the anatomical shape, for example, of the vascular ducts, while still being a sufficiently rigid structure to allow elastically recovering the rectilinear position when not stressed by the curvilinear duct wall.

Therefore, it is required that the guide wire is capable of allowing a flexibility thereof, which is suitable to allow a bending of the body thereof, or a torsion that is necessary to navigate through the patient's anatomical structure and, in some cases, to minimize the injury induced by the device passage through the ducts, for example, vascular ducts. Nonetheless, it is also convenient in some applications that the medical device is sufficiently rigid in order not to collapse, for example, when navigating through a relatively wide vascular duct. Furthermore, it is convenient for this medical device to have a relative sufficient torsional rigidity in order to allow an accurate control of the rotation thereof around the longitudinal axis thereof, when manoeuvred through the proximal end thereof, in order to transmit the movement to the apical portions thereof, in order to, for example, negotiate the insertion of the device apical end into a bifurcation side branch, for example, a vascular one, or in order to by-pass any obstacles present in the ducts.

Another characteristic which these devices need to have is to minimize the friction action that this device exerts against the duct walls in which it is inserted, in order to facilitate the insertion and removal thereof.

In addition, it is convenient that these devices result to be particularly resistant and durable in order to ensure a complete removal thereof at the end of the surgery.

In particular, the percutaneous treatment of lesions in the proximity of the coronary bifurcations, or Bifurcation Lesions BLs, constitutes a particular problem, both clinic intraoperatory and long-term, due to the difficulty that the operator experiences when implanting one or more stents into the different types of lesion, maintaining the patency of all the branches, while achieving the ideal covering of the whole lesion by the stents, while avoiding that the lesion or restenosis arises again.

This type of lesions in the proximity of the bifurcations results to be extremely heterogeneous in the anatomical presentation thereof, both the incidence angle with which the side branch takes its origin (for example, it can be an nearly 90° branch, known by the term “T”-type, or a V-branch, known by the term “Y”-type BL), and the involvement in the lesion of the three segments composing the BL (known by the definition 1-4c-type BL) being variable:

1. lesion passing through the main vessel proximal length;
2. lesion affecting the main vessel distal length;
3. lesion affecting the side branch.

To date, the known guide wires for the treatment of coronary vessels consists in a semirigid, long, linear length, and a short, more flexible, or “floppy” apical length which is adapted to prevent vascular lesions during the guide wire advance inside the vessels, which apical length can be more or less pre-bent at the distal end thereof, at the apex, in order to negotiate the access to the bifurcation desired branch.

A catheter, or stent delivery system SDS, is then advanced, which “rides” along the guide wire to the level of the vascular segment to be treated, by running along the already positioned the guide wire, to the surgical site.

In the case of lesions at bifurcations, or BLs, it is necessary to locate 2 guide wires: one in the main vessel, and a second one in the side branch. Then, balloon catheters are advanced along these guide wires in order to widen the lesions, thus making the successive correct positioning of the stent delivery systems SDSs easier.

However, the current guide wires, while being satisfactory under many points of view, present considerable drawbacks, particularly when used in the proximity of the bifurcations.

The commonest drawback when using these known guide wires consists in the twisting, or tangling, or crossing of the two guide wires which, distally twisting around each other, prevent the advance of the catheters to the lesion level.

This drawback statistically accounts for 25-30% of the treated cases, and it is solved by maintaining the catheter at the reached position, then withdrawing one of the two guide wires into the lumen which the catheter rides the guide wire with, then advancing it again into the vessel, while hoping that, upon doing the ride again, it does not wind again on the other guide wire. This manoeuvre involves a temporary loss of the approach to the vessel which is intended to be treated, which, in the case of a dissection, could be difficult to obtain again, if not impossible, also with a risk of complications during the procedure.

When stent delivery systems SDSs are used which are not dedicated to the treatment of bifurcations, it is necessary, after the implant of a first endoprosthesis, or stent, to laterally run the meshes of this stent again, in order to reach the side branch again, to complete the treatment of the lesion.

The most used technique to facilitate this stent recrossing is the technique called “Jailing Wire”.

This technique consists in leaving a guide wire into the side branch during the stent implant in the main vessel. In this manner, the second guide wire results to be jailed by the stent, but it allows visualizing the side branch origin and the travel thereof, also when this branch should be obstructed as a consequence of the dislocation of the plaque material dislocated by the stent arranged in the main vessel, or in the case of a dissection following the stent implant into the main vessel.

In this case, the Jailed Guide Wire, or stent-jailed guide wire, should facilitate the approach to the lumen (FIG. 6 and FIG. 7).

Once reached again the side branch lumen with a new guide wire, the jailed guide wire has to be removed. However, this manoeuvre poses the risk of a guide wire rupture at the floppy portion thereof, and anyway always involves a considerable traction of the stent and the treated or stented vessel. Generally, this manoeuvre does not cause undue problems, but it anyhow involves stretching actions of the vessel segment downstream the stent and at the side branch mouth, as well as, sometimes, a “curling up” of the stent in the proximal length, where the vessel wall also could be damaged.

Furthermore, in the case drug eluted stents are used, the abrasive action of the guide wire on the medicated stent outer wall could impair the efficiency thereof.

From what has been stated herein above, it is inferred that the difficulty in treating lesions in the proximity of bifurcations, the results of which largely depend on the operator's skill, which lead to long, difficult, and often complicated surgeries, with even doubtful results, not being able to allow maintaining a continuous approach to both bifurcation vessels.

In order to partially obviate these drawbacks, catheters or stent delivery systems SDSs dedicated to the treatment of the bifurcations have been proposed.

These are usually systems which ride or advance on both guide wires which reach both bifurcation branches. Some of these prior art solutions have bifurcated, or double, or side-by-side balloons, or individual balloons with at least two lumens for the guide wire remaining in the main vessel and the guide wire which is introduced into the side branch. In such manner, the approach is continuous during the whole surgical procedure, and there is no need to pass through the implanted stent meshes again.

The limitations of the dedicated stent delivery systems SDSs are most of all the difficult positioning and the device unsuitable rotation, that is, the impossibility to rotate the stent delivery system SDS so as to position the side port, useful for the passage of the side branch guide wire, just in front of the side branch ostium.

In the case where the guide wire positioned in the side branch has a high flexural rigidity, it could happen that the dedicated stent delivery system SDS is not able to reach the side branch ostium with the side port (FIG. 1).

Instead, in the case the guide wire results to be excessively flexible, for example, because the floppy length has been maintained astride the bifurcation, the stent delivery system SDS, when pushed by the operator, could deform the floppy part of the guide wire inserted in the side branch so as to fold it on itself in the main vessel and making so that the side port passes past the side branch ostium, to finally positioning itself in the main vessel distal length (FIG. 2). In this latter case, the optional stent expansion or dilatation would lead to a very dangerous U-jailing of the guide wire distal portion, and in practice to an impossibility to recover the guide wire without large risks of a guide wire rupture, a vessel length lesion, and a strong abnormal deformation for the stent.

However, considerable drawbacks have been noticed also in the case of a correct positioning of the guide wire inside the side branch, due to the guide wire arrangement into the bifurcation. In fact, the guide wire which is arranged in the side branch, is arranged on a plane formed by the main vessel and the side branch, and the bending thereof when it reaches the apical portion of the side branch vessel wall leads to a curvature radius which of course results to be the widest possible, due to the elastic recovery of the guide wire in the rectilinear position and the actions by the vessels walls which oppose the guide wire advance thrust (see FIGS. 3, 4, and 5). The guide wire that is going to be inserted, and is introduced, into the side branch exerts a force upon the side branch ostium portion, and consequently on the opposite main branch inner surface, which actions are proportional to the guide wire flexural rigidity, which tends to widen the bifurcation angle.

When the stent delivery system comes in the proximity of the bifurcation, the side branch guide wire tends to push the stent delivery system SDS against the main vessel wall opposite the side branch ostium, increasing the friction between the device and the vessel inner walls. In this manner, due to this increased friction, the stent delivery system SDS further results to be even more difficult to be correctly positioned.

Furthermore, the stent delivery system SDS rotation to bring the guide wire side port to face the side branch ostium, results to be made easier only when the system comes to the proximity of the bifurcation, when it is initially already suitably rotated towards the side branch ostium (FIGS. 8 and 9). Instead, in the case where the side opening results to be angularly rotated of an angle above 90° relative to the side branch ostium (see FIG. 9), laterally entering the stent delivery system side port, the guide wire exerts an adverse tangential thrust which tends to rotate the stent delivery system SDS in a direction which is opposite to the correct rotation. This torsional force is the resultant of the sum of the advancing thrusting action applied by the operator on the stent delivery system SDS proximal part and the bending resistance action of the guide wire, which is tangentially applied. The higher the incidence angle of the stent delivery system side port relative to the side branch plane, the higher the adverse contra-rotational effect.

### OBJECT OF THE INVENTION

Therefore, the object of the present invention is to provide a guide wire which is able to obviate the drawbacks of the prior art, particularly when used in the treatment of lesions at bifurcations.

These and other objects are achieved by means of a guide wire according to claim 1.

In accordance with a general embodiment, a navigation guide wire through an anatomical apparatus, for example, a vascular system, comprises:
an elongated body having a proximal end, a distal end, and a longitudinal extension along an axis extending at least from said proximal end to said distal end;
at least one first proximal portion having a predetermined first flexural rigidity capable of a first predetermined flexure in at least one plane comprising at least one portion of said longitudinal axis in order to allow a first curvature at said proximal portion when bending stressed, while ensuring a predetermined elastic return in a rectilinear position of the elongated body proximal portion when not subjected to stresses;
at least one second distal portion having a predetermined second flexural rigidity, the amount of which is equal to or higher than said first flexural rigidity of said at least one first proximal portion, capable of a predetermined flexibility of said second distal portion in order to obtain a second curvature when bending stressed in at least one plane comprising at least one portion of said longitudinal axis, while ensuring an elastic return in a rectilinear position of said distal portion of said elongated body when not subjected to stress;
at least one intermediate length, arranged between said first proximal portion and said second distal portion, having a third flexural rigidity essentially lower than said first flexural rigidity and said second flexural rigidity, which allows a predetermined flexibility capable of a third curvature essentially more pronounced compared to said first curvature and second curvature, so as to allow a considerable direction change between said first proximal portion and said second distal portion.

In accordance with a further general embodiment, in a guide wire said third flexural rigidity of said intermediate length results to be essentially lesser than said first flexural rigidity and said second flexural rigidity only in a first predetermined plane comprising at least one portion of the longitudinal axis of said intermediate length, said intermediate length having, in a second plane orthogonal to said first plane, a fourth flexural rigidity essentially higher than said third flexural rigidity, which allows a predetermined reduced flexibility capable of a less pronounced fourth curvature than said third curvature in said fourth orthogonal plane.

In accordance with a further general embodiment, in a guide wire, distally to said distal portion, a further distal or apical portion is provided, which has a higher flexibility than said second distal portion, so as to be flexible or floppy, so as to result to be adapted to negotiate even tortuous branches, for example, in a vascular system.

With the above-mentioned embodiments and the exemplary embodiments set forth below, several advantages are achieved, and, in particular:
a medical device which results to have an easy manoeuvrability and a quick torsional feedback, while being extremely flexible in a localized intermediate portion;
a guide wire capable of advancing to place, arranging itself in practice automatically astride the bifurcation, with the distal portion inserted into the side branch, thus avoiding undesired stresses to the vessels walls;
a guide wire capable of being inserted inside vessels having particularly reduced dimensions, avoiding keeping guide wires lengths having particularly high flexural rigidities astride the bifurcation, which on the contrary would pose a risk of damage for the bifurcation inner walls;
a correct approach for the dedicated stent delivery systems SDSs provided with side ports for the guide wires of the side branches, allowing the stent delivery system SDS side port to perfectly face the side branch ostium;
a guide wire which locally receives small curvature radiuses, and allows the treatment of “T”-type bifurcation having a very pronounced angle;
a guide wire capable of drastically reducing the advance resistance of the stent delivery system SDS, avoiding rubbing actions between the stent delivery system SDS and the vessel inner wall;
a complete elimination of the adverse rotation produced on the stent delivery system SDS by the guide wire.

These and other objects and advantages are achieved by the embodiments given below, and the further embodiments are amenable to the annexed dependant embodiments exemplified herein below.

### BRIEF DESCRIPTION OF THE DRAWINGS

The technical features of the invention, according to the above-mentioned objects, are traceable from the contents of the above-mentioned embodiments, and the advantages thereof will result more clearly in the following detailed description, given with reference to the annexed drawings, which illustrate a merely exemplary, non-limiting embodiment thereof, and in which:

FIG. 1 shows a bifurcation seen in section, in which two guide wires are inserted into the main duct and into the side branch, respectively, astride which a stent delivery system SDS is located according to a conventional technique;

FIG. 2 illustrates a bifurcation seen in section in which two guide wires are inserted into the main duct and into the side branch, respectively, and a stent delivery system SDS is advanced until entrapping a portion of the side branch guide wire;

FIGS. 3, 4, and 5 show a bifurcation seen in section in which a guide wire is present in the main duct, and a guide wire is gradually advanced thereby entering the side branch, thus abutting against the walls defining the side branch ostium and the opposite main branch inner wall;

FIGS. 6 and 7 show a bifurcation seen in section and the same bifurcation in a sectional axonometric view, in which two guide wires are located in the main duct and in the side branch, respectively, and a conventional stent delivery system SDS is advanced only on the guide wire which is arranged in the main duct until locking the guide wire arranged in the side branch along the vessel walls at the bifurcation;

FIGS. 8 and 9 show in a sectional axonometric view and in a sectional transversal view a bifurcation in which there are one guide wire in the main duct and one in the side branch, both of which are covered by a dedicated stent delivery system SDS put astride them, where the side branch guide wire comes in through a stent delivery system SDS side port and, according to the approach position to the bifurcation, it exerts a torsional rotation on the stent delivery system SDS due to the flexural rigidity thereof;

FIG. 10 shows a guide wire according to the invention provided with a floppy apical portion, a second distal portion, a flexible intermediate portion, and a first proximal portion, as well as an optional torsional and thrust control device of the guide wire;

FIG. 11 shows in axonometric view a detail of a guide wire at the intermediate length;

FIG. 12 illustrates in a side view a detail seen in section of the guide wire at the intermediate length connecting the first proximal portion and the second distal portion;

FIG. 13 shows in a sectional side view a main duct bifurcation in a side branch in which a first guide wire is arranged in the main duct, and a second guide wire is arranged in the side branch;

FIG. 14 illustrates a cross-section of a main branch bifurcation in a side branch, in which a first guide wire is arranged in the main branch, and a second guide wire is arranged in the side branch, both covered by a dedicated stent delivery system SDS put astride them, which is arranged with the side port thereof facing the side branch ostium;

FIG. 15 shows in a partially sectional axonometric view a further embodiment of a guide wire according to the invention;

FIG. 16 shows a sectional side view of a detail of the intermediate portion of the guide wire in FIG. 15;

FIG. 17 illustrates the guide wire of FIG. 15 in which the intermediate length is bent in order to bring the second distal portion to a different angle as compared to the arrangement of the guide wire first proximal portion;

FIG. 18 shows in a partially sectional axonometric view a guide wire which is not subject to external stresses according to a further embodiment;

FIG. 19 shows a sectional side view of the intermediate length of the guide wire in FIG. 18;

FIG. 20 illustrates the guide wire of FIG. 18 in which the guide wire second distal portion is angularly arranged relative to the guide wire first proximal portion, thus forming an accentuated curvature of the intermediate length portion;

FIG. 21a shows in a partially sectional axonometric view a guide wire according to a further embodiment;

FIG. 21b shows in a sectional side view a guide wire according to a further embodiment;

FIG. 22 shows in a sectional side view an intermediate length of a guide wire according to a further embodiment;

FIG. 23 illustrates in a sectional axonometric view according to the line XXIII-XXIII of FIG. 22 the guide wire intermediate portion;

FIGS. 24 to 33 show a in sectional side view or in a sectional axonometric view eight different embodiments of the intermediate length between a first proximal portion and a second distal portion of the guide wire;

FIG. 34 shows in a sectional axonometric view an intermediate portion of a guide wire provided with a reinforcing member;

FIG. 35 illustrates in an axonometric view a reinforcing member for a guide wire intermediate portion;

FIG. 36 shows in a partially sectional side view a guide wire provided, in the intermediate portion thereof, with a reinforcing member;

FIG. 37 illustrates in an axonometric view a reinforcing member for a guide wire intermediate length;

FIG. 38 shows in a sectional side view a guide wire intermediate length provided with a protective member according to an embodiment;

FIG. 39 illustrates in a sectional side view a guide wire intermediate length provided with a sheath protective member;

FIG. 40 shows in a sectional side view a guide wire intermediate length comprising a coiled spring member;

FIG. 41 illustrates in a partially sectional side view a guide wire provided, on the intermediate length thereof, with a protective sheath;

FIG. 42 illustrates in a sectional side view a guide wire comprising a first proximal portion having a coiled spring, and a second distal portion having a coiled spring, as well as an intermediate length also comprising a coiled spring having a different and lower elastic constant relative to the first and second coiled springs of the proximal and distal portions;

FIG. 43 illustrates in a sectional side view a guide wire with tubular elongated body in the portion of the intermediate length thereof internally provided with a coiled spring member;

FIG. 44 illustrates a guide wire with tubular elongated body in the intermediate length thereof internally provided with a synthetic material filling member;

FIG. 45 illustrates in an axonometric view a guide wire portion where the guide wire body is composed of a tubular element and has, in the intermediate length thereof, at least one notch which in the Figure results to be stressed at the small bridging portion which connects the proximal and distal portions, leading to the opening thereof, and to a differently angulated arrangement of the first proximal portion relative to the second distal portion;

FIG. 46 shows the guide wire of FIG. 45 in a rectilinear position;

FIG. 47 illustrates the cross-section of a guide wire according to the line XLVII-XLVII, or 47-47, of FIG. 46;

FIG. 48 shows a cross-section of a guide wire in which a reinforcing sheet is present;

FIG. 49 shows a guide wire section with reinforcing sheet having a different sectional geometry;

FIG. 50 shows a sectional side view of the intermediate length of a tubular guide wire with reinforcing sheet;

FIGS. 51, 52, and 53 show a stent delivery system sided by a tubular device having a floppy apical portion, a second essentially flexurally rigid distal portion, a first essentially flexurally rigid proximal portion, and an intermediate length between said first and second distal and proximal portions which is essentially flexible and capable of small curvature radiuses;

FIG. 54 shows in a sectional side view a guide wire with tubular elongated body internally provided with a stiffening cable;

FIGS. 55 and 56 illustrate a top and side view of a guide wire intermediate length provided with hinges;

FIG. 57 shows a guide wire intermediate length comprising a hinge provided, on a portion thereof, with prongs which receive therebetween an extension of the opposite hinge portion;

FIG. 59 illustrates in a sectional, exploded, axonometric view a guide wire intermediate length provided with a hinge;

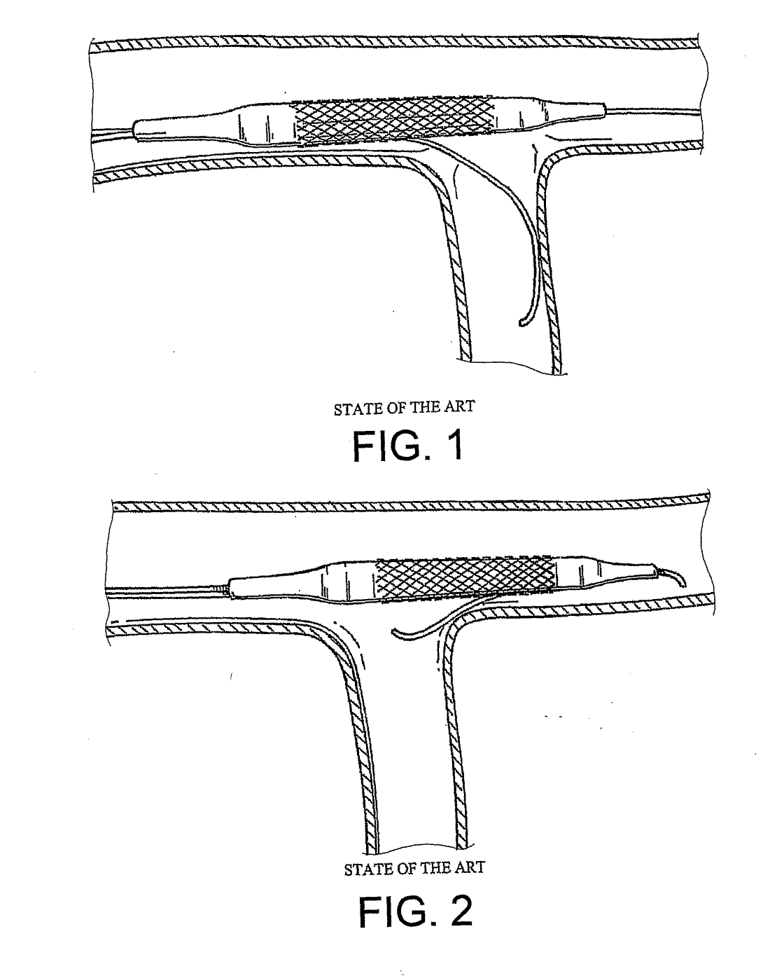

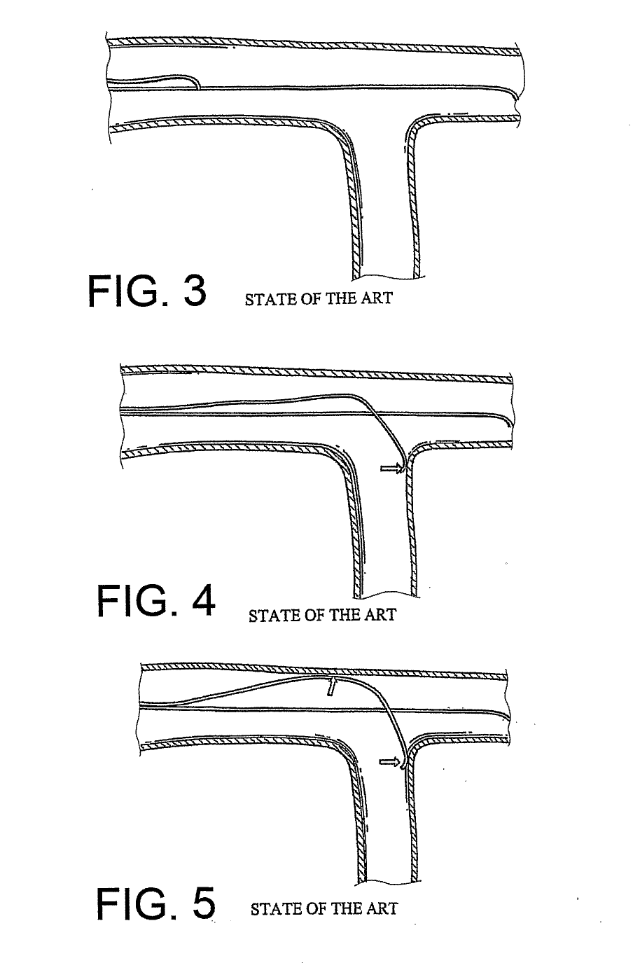

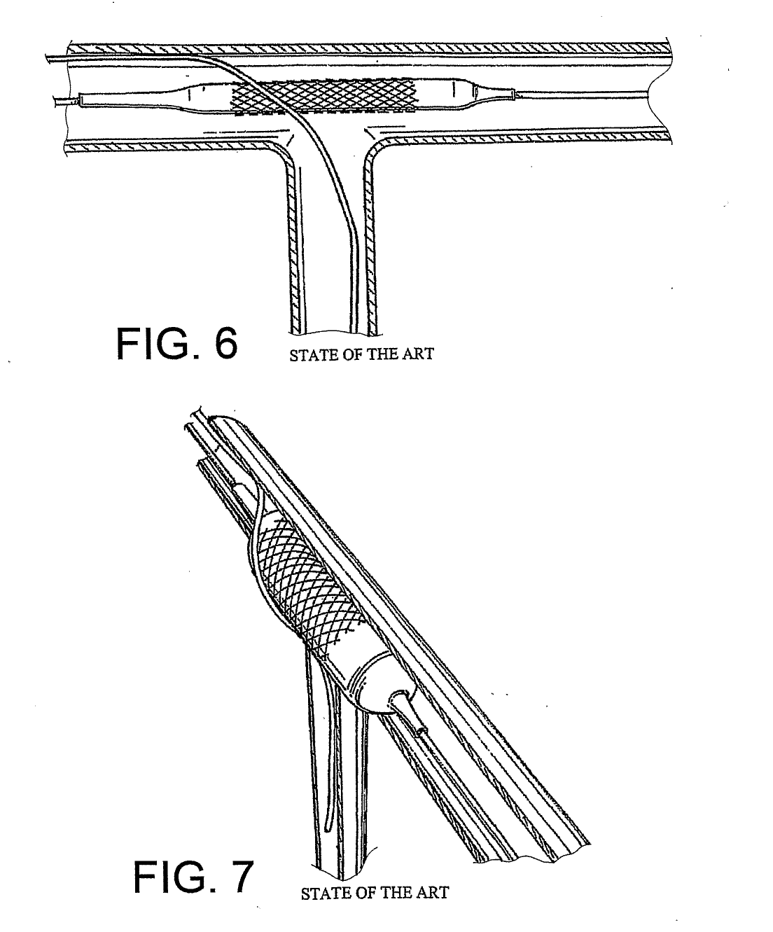

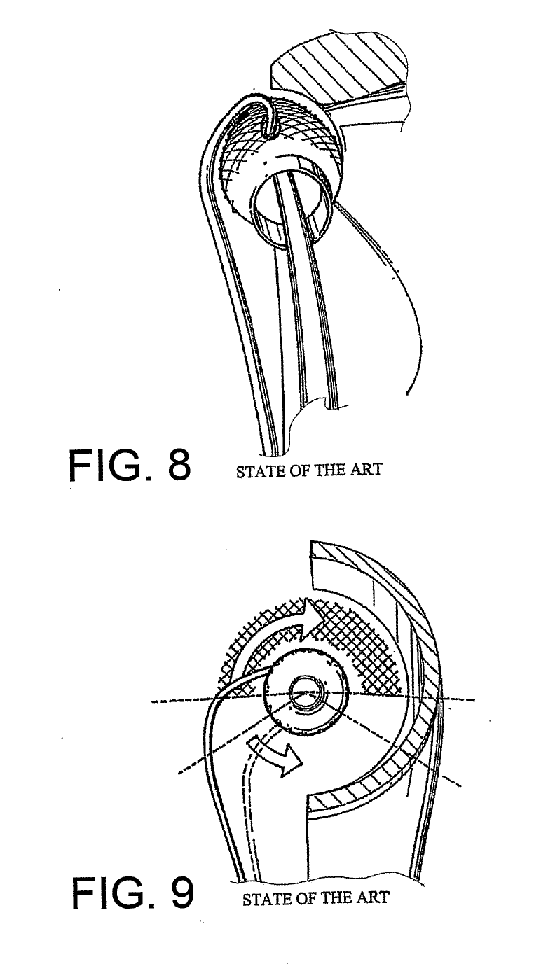

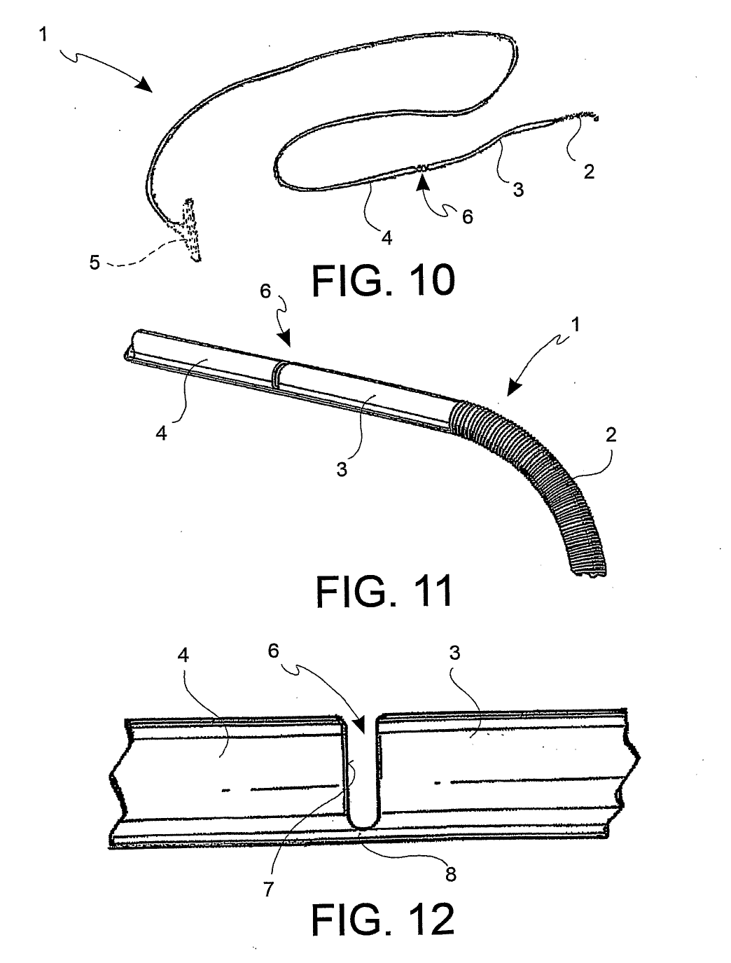

FIG. 58 shows a guide wire intermediate length comprising two hinges;

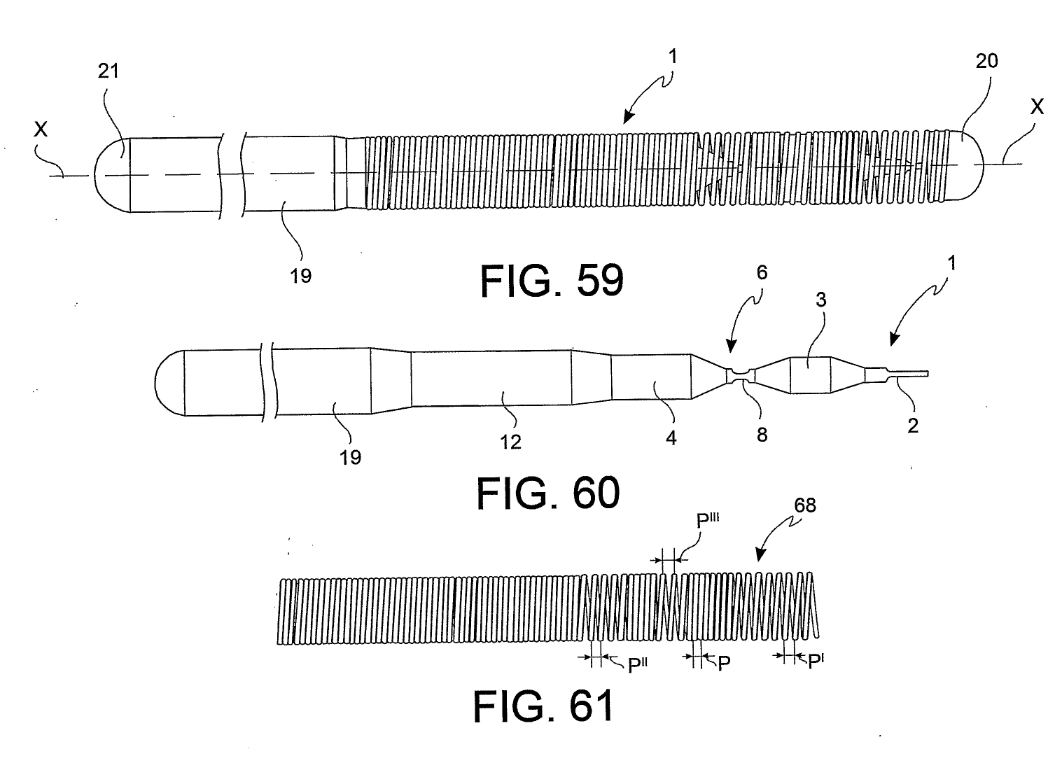

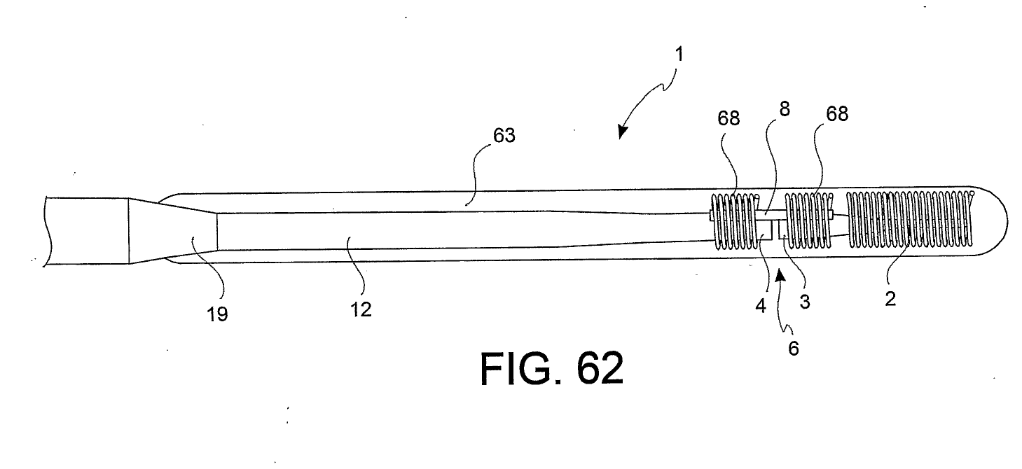

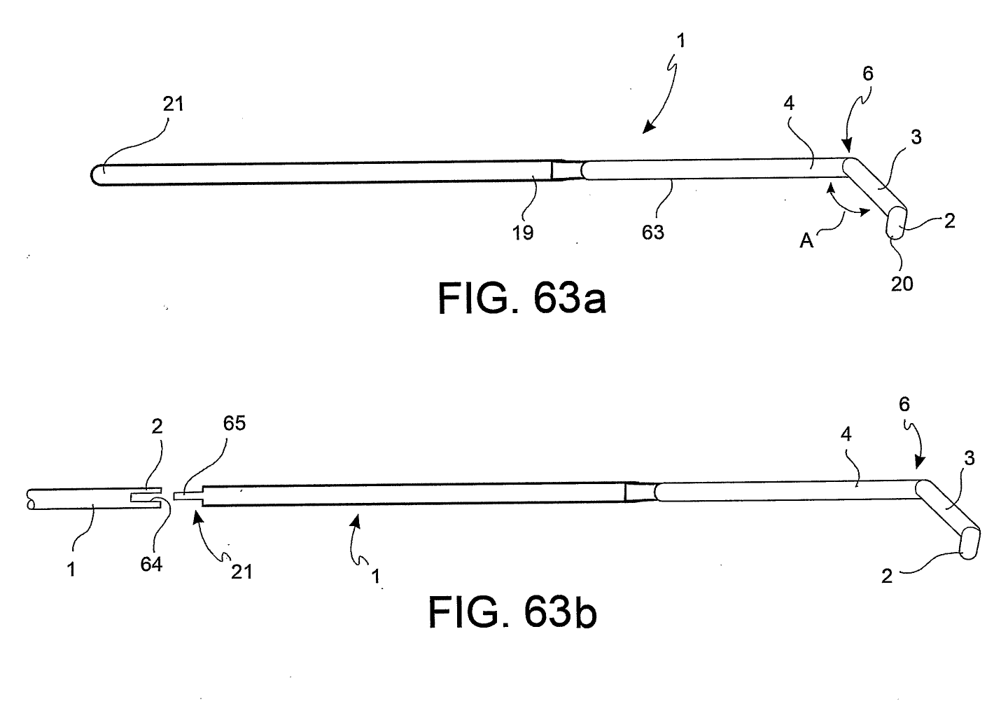

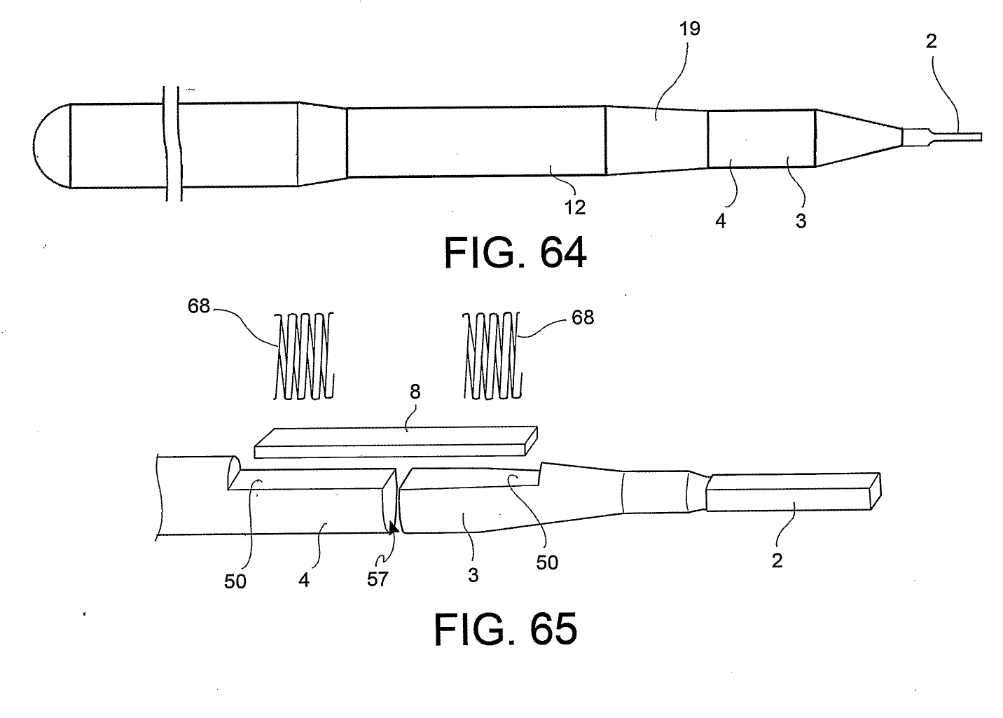

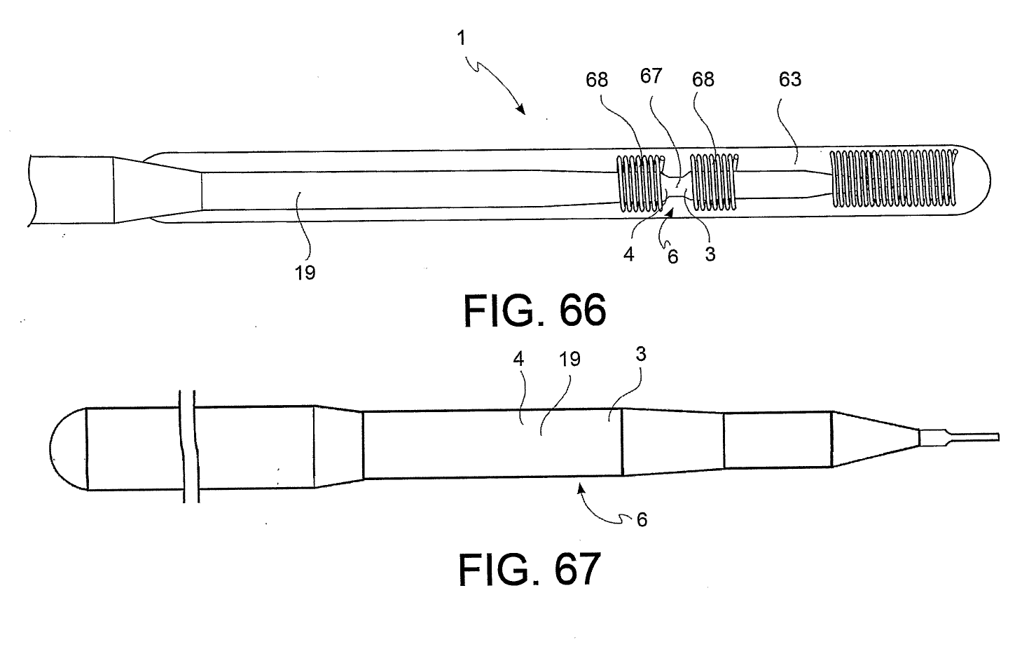

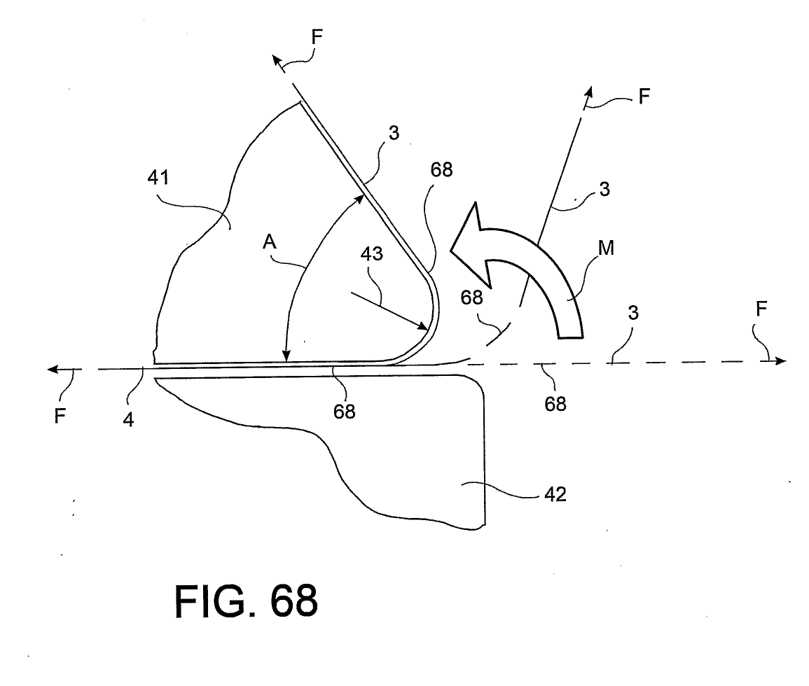

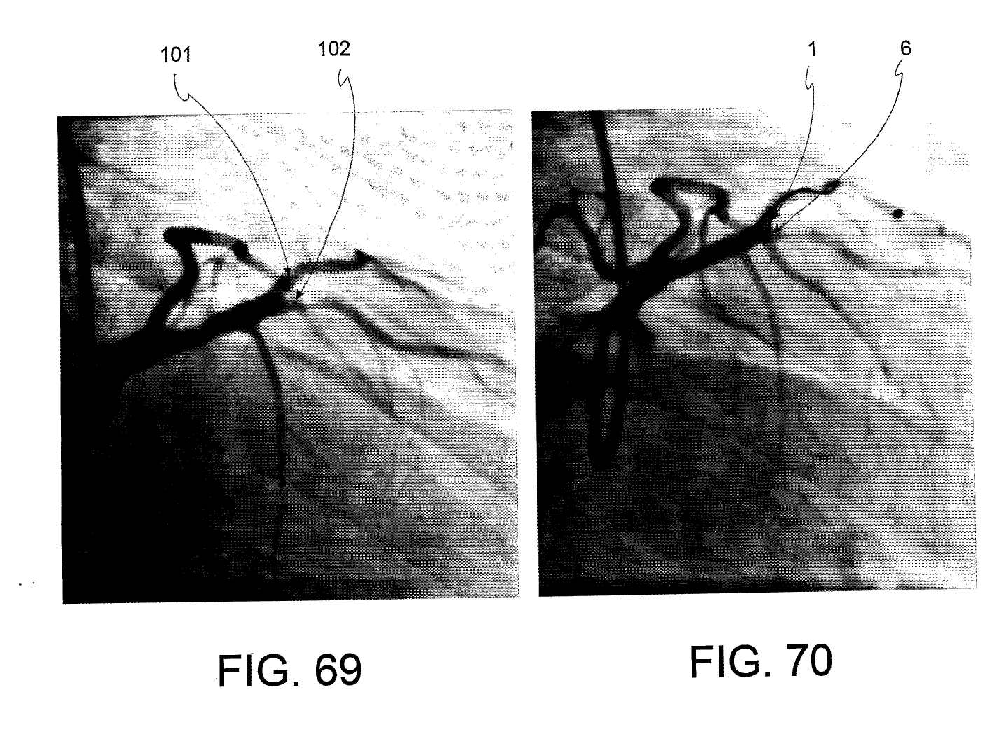

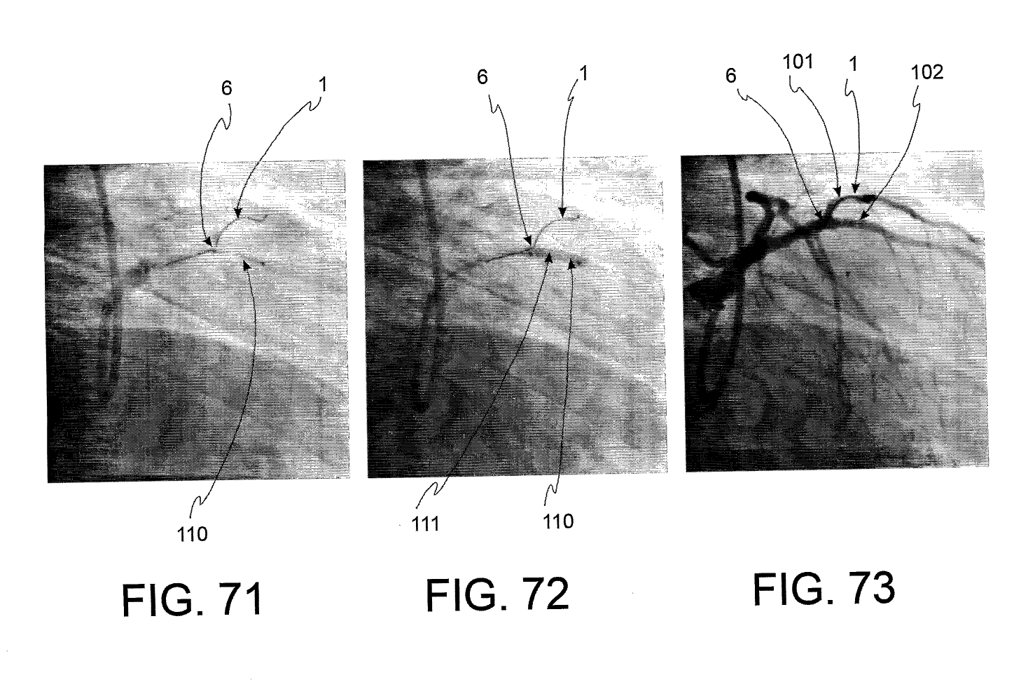

FIG. 59 illustrates in a side view portions of a guide wire according to a further embodiment, with body or core or core wire and a covering of at least a distal part thereof by means of a helical wound wire;

FIG. 60 represents portions of the body or core wire of the guide wire in FIG. 59;

FIG. 61 shows a side view of the helical wire provided on the guide wire in FIG. 59;

FIG. 62 illustrates a portion of a guide wire according to a further embodiment with body or core wire on which a plurality of helical-wound wire lengths is provided and a hinge-like portion thereof and an outer covering of the at least one more distal length;

FIG. 63a illustrates a side view of a guide wire according to a further embodiment with a hinge-like portion and a more distal folded portion;

FIG. 63b illustrates a partially sectional side view of a guide wire with a distal end suitable to be coupled to a further extension guide wire, such as by means of a DOC guide wire extension coupling;

FIGS. 64 and 65 illustrate side views of partially sectioned portions of a body for a guide wire wherein FIG. 64 shows the body before processing and FIG. 65 a portion of the body after cutting and levelling to provide the facing portions of the guide wire hinge-like portion;

FIG. 66 shows a partially sectional side view of a guide wire according to a further embodiment comprising a body or core wire to which a plurality of helical-wound wires is applied, which proximally define a hinge-like length;

FIG. 67 is a partially sectional side view of the core wire of guide wire in FIG. 66 prior to the provision of the hinge-like length;

FIG. 68 illustrates steps of the method for providing the hinge-like length for the guide wire in FIG. 66;

FIG. 69 illustrates an image of a radioscopy or scopy carried out in-vivo during a test in which the presence of a length of a vessel with Y-bifurcation having a partial occlusion proximate to the ostium of a side branch can be seen;

FIG. 70 illustrates an image of a radioscopy of the same region as in FIG. 69 in which two guide wires are present, which are inserted in a same branch of the Y-bifurcation, one of these guide wires being manufactured according to the state of the art and a second guide wire being manufactured according to the invention, with the hinge arranged proximate to the ostium of the other branch of the Y-bifurcation which has the occlusion;

FIG. 71 illustrates an enlarged view of FIG. 70 where the guide wire that reaches the not-occluded branch of the Y-bifurcation along with a stent-loaded catheter to dilate and impalcare the plaque dislocated to reconstitute the lumen still in the collapsed position and perfectly aligned with the ostium of the occluded branch exit the guide wire catheter;

FIG. 72 illustrates the catheter in FIG. 71 expanded;

FIG. 73 illustrates the same enlarged view as in FIGS. 71 and 72 in which after the stent has been placed, the catheter has been deflated and withdrawn along with the guide wire while the contrast fluid injected in the vessels shows the total recovery of the patency of the Y-bifurcation branch that was occluded.

### DETAILED DESCRIPTION OF SEVERAL PREFERRED EMBODIMENTS

In accordance with a general embodiment, a guide wire 1 comprises an elongated body 19 having a distal end 20 and a proximal end 21. Said body has a longitudinal extension along an axis X-X extending at least from said proximal end 21 to said distal end 20.

The guide wire 1 has at least one first proximal portion 4, a second distal portion 3 and an intermediate portion or intermediate length 6.

Advantageously, said at least one first proximal portion 4 has a predetermined flexural rigidity Kf1 which is capable of a first predetermined curvature of said at least one first proximal portion in at least one plane, allowing a first curvature when said first proximal portion is bending stressed, while allowing an elastic return in the rectilinear position when not subjected to stresses.

Said at least one second distal portion 3 has a predetermined second flexural rigidity Kf2 which advantageously results to be of an amount which is equal to (Kf1=Kf2) or higher (Kf2>Kf1) than said at least one first proximal portion 4 of the elongated body 19, so as to allow a predetermined flexibility of the second distal portion, thereby allowing a curvature of said portion when subjected to a bending stress, while allowing an elastic return in the rectilinear position of the elongated body distal portion when this is not subjected to stress.

With particular advantage, at least one intermediate length 6 is provided, which is arranged between said first proximal portion 4 and said second distal portion 3. Said intermediate length 6 has an essentially lesser third flexural rigidity Kf3 (Kf3<Kf1 and Kf3<Kf2) than said first flexural rigidity Kf1 and said second flexural rigidity Kf2, which third rigidity Kf3 allows a predetermined flexibility capable of an essentially third curvature which is more pronounced or capable of smaller radiuses than said first curvature and second curvature, so as to allow a substantial direction change between said first proximal portion and said second distal portion.

In accordance with an embodiment, said third flexural rigidity Kf3 of said intermediate length results to be essentially lesser than said first flexural rigidity and second flexural rigidity only in a first predetermined plane comprising at least one portion of the longitudinal axis of said intermediate length 6, said intermediate length having in a second plane, orthogonal to said first plane, an essentially higher fourth flexural rigidity Kf4 (Kf4>Kf3) than said third flexural rigidity, which allows a predetermined reduced flexibility capable of a less pronounced fourth curvature than said third curvature in said fourth orthogonal plane.

In accordance with an embodiment, distally to said distal portion 3, a further distal or apical portion 2 is provided, having a higher flexibility than said second distal portion 3, so as to turn out to be flexible or floppy, and so as to turn out to be suitable to negotiate even tortuous branched, for example, in a vascular system.

In accordance with an embodiment, said intermediate length 6 comprises at least one notch 7, 17, 27, 37, 47 transversal to said elongated body 19 suitable to define a small bridge 8 between said first proximal portion 4 and said second distal portion 3.

In accordance with an embodiment, said notch 57 is a complete cut of the elongated body 19 of the guide wire 1 and the small bridge 8 is a portion added to said body which connects the first proximal portion 4 to the second distal portion 4. Advantageously, said small bridge 8 is a portion of a body and/or an element added to the body which connects the proximal and distal portions to each other. In accordance with an embodiment, only part of the facing distal and proximal ends of the distal and proximal body portions are connected to each other, particularly for example by means of a small bridge 8 arranged proximate to a portion of the outer surface of said elongated body 19. In accordance with an embodiment, the body length suitable to receive the small bridge is previously leveled to form in the distal end of the first proximal portion 4 a first seat or proximal seat for the small bridge 60 and in the proximal end of the second distal portion 3 a second seat or distal seat for the small bridge 61.

Said small bridge 8 is then coupled to the first seat 60 of the first proximal portion 4 and to the second seat 61 of the second distal portion 3 of the wire 1.

In accordance with an embodiment, said small bridge is fasciato or bonded to the elongated body for example by means of a coil wound wire which, in turn, in accordance with an embodiment, is welded with the small bridge to the elongated body.

In accordance with an embodiment, said intermediate length 6 comprises a plurality of notches 7, 17, 27, 37, 47 transversal to said longitudinal axis, for example forming concavities facing the same side of said elongated body 19 which provide a plurality of small bridges 8.

In accordance with an embodiment, said at least one notch has in the longitudinal section a U-shaped form 7 or a V-shaped form 37 or a circular-profile form 27 or counter-posed V-shaped form with openings facing opposite sides of the elongated body such as to leave a small bridge 8 in the middle of said longitudinal section (FIGS. 28, 30, 31 and 59 and 60). Preferably, in accordance with an embodiment, relieves 15 are provided which define in said form of the longitudinal section of the notch 7, 17, 27, 37, 47 longitudinally elongated slots to form at the notch end at least one small bridge 8 having a greater longitudinal extension than the minimum aperture of the form of said notch in the longitudinal direction.

In accordance with an embodiment, said intermediate length 6 comprises an outer covering element 18, for example a spiral element or coil or coil wound wire 68 is arranged about the elongated body 19 or core wire in at least one length of the elongated body, for example about the distal length. In accordance with an embodiment said coil wound wire or coil is arranged about said intermediate length 6 and particularly astride said at least one notch 7 and such as to cover the latter. In accordance with an embodiment, said coil wound wire is arranged just before and just after said intermediate length 6.

In accordance with an embodiment, a sheath, for example an elastic sheath 29, is arranged about at least one length of the elongated body 19, for example about at least said intermediate length 6 astride the notch 7 and such as to cover the latter, preferably as a jacket, about an entire distal length. In accordance with an embodiment, said intermediate length 6 comprises an outer covering element, for example a polyurethane layer or jacket.

In accordance with an embodiment, a reinforcing member 16 is provided astride said intermediate length 6, comprising at least one annular portion arranged around the distal end of said first proximal portion 4 and/or around the proximal end of said second distal portion 3, and a length having a longitudinal extension which is adapted to arrange itself in the proximity of said at least one small bridge 8 defined by said at least one notch 7.

In accordance with an embodiment, the longitudinal extension of said at least one transversal notch 7 results to be lower than the transversal dimension to the longitudinal axis of the elongated body or depth of said at least one transversal notch.

In accordance with a further embodiment, the longitudinal extension of said at least one transversal notch 17 results to be greater than the transversal dimension at the longitudinal axis of the elongated body or depth of said notch.

In accordance with an embodiment, said intermediate length 6 comprises a coiled member 48, or coil, connecting the distal end of said first proximal portion 4 to the proximal end of said second distal portion 3. In accordance with an embodiment, said coiled member 48 results to be the only connection between the proximal portion 4 distal end and the distal portion 3 proximal end. Said coiled member 48 has an elastic constant Kf3 which is lesser or highly lesser than that Kf1 of the proximal portion 4 and that Kf2 of the distal portion 3. In accordance with an embodiment, also the proximal 4 and distal 3 portions comprise coiled or spring members having higher elastic constants (Kf3<Kf1 and Kf3<Kf2, or Kf3<<Kf1 and Kf3<<Kf2). Advantageously, said coil wound elements or wires are arranged about the elongated body 19 or alternatively they are the only connection between lengths of the guide wire body.

In accordance with an embodiment, said elongated body comprises a tubular body 150.

In accordance with an embodiment, said tubular body 150 comprises, at least in the intermediate length 6, an elastic filling member, for example, a coiled spring 300 and/or synthetic material 400, and/or an outer protective sheath 28.

In accordance with an embodiment, a longitudinal reinforcing sheet 500 is provided, which is arranged in the tubular body 150 wall at least in the intermediate length 6.

In accordance with an embodiment, a wire 200 is provided within said tubular body 150.

In accordance with an embodiment, a stent delivery system 700 is placed beside a tubular device 600 having a floppy apical portion 602, a second distal portion 603, substantially flexurally rigid, a first proximal portion 604, substantially flexurally rigid, and a substantially flexible intermediate length 606 between said first and second distal and proximal portions, which is susceptible of small bending radii.

In accordance with an embodiment, a guide wire with extended tubular body is internally provided with a stiffening cable 200.

In accordance with an embodiment, an intermediate length 6 of a guide wire 1 is provided with hinges 800. In accordance with an embodiment, an intermediate length 6 of a guide wire comprising a hinge 800 provided with prongs on a portion thereof.

In accordance with an embodiment, an intermediate length of a guide wire provided with a hinge 800, whereas in accordance with a further embodiment, an intermediate length of a guide wire comprising two hinges 800.

It should be understood that the solution will be able to take, in its practical implementation, also shapes and configurations other than the one which has been illustrated above, without for this departing from the present scope of protection. Furthermore, all the elements will be able to be replaced by technically equivalent elements, and the shapes, dimensions, and the materials in use will be able to be any one, according to the needs.

In accordance with an embodiment, a guide wire 1 has distally to said distal portion 3 at least one further distal portion 2, having a higher flexibility than said second distal portion 3, such as to result flexible or floppy, and such as to result suitable to negotiate even tortuous branches, for example, in a vascular system.

In accordance with an embodiment, proximally to said proximal portion 4 at least one further proximal portion 12 is provided, which advantageously has at least one fifth flexural rigidity Kf5 substantially higher than said first flexural rigidity Kf1. Preferably, a plurality of further proximal portions 12 are provided which have increasing flexural rigidity as more proximal portions are being formed and the proximal end 21 is being approached.

In accordance with an embodiment, said intermediate length 6, or hinge, comprises at least one notch 7, 17, 27, 37, 47, 57, 67 transversal to said elongated body 19 which co-operates with a small bridge 8 connecting said first proximal portion 4 to said second distal portion 3, particularly, though not necessarily, a small bridge arranged proximate to a portion of the outer surface of said elongated body.

In accordance with an embodiment, said intermediate length 6 comprises at least one notch 57 transversal to said elongated body 19 which forms a partition separating said first proximal portion 4 from said second distal portion 3.

In accordance with an embodiment, said first proximal portion 4 is connected to said second distal portion 3, separated from the first portion 4 by the cut 57, by means of a small bridge 8.

In accordance with an embodiment, said small bridge 8 is a rectangular section ribbon bridge, and preferably said small bridge is coupled to flattenings 50 provided at the distal end of the first proximal length 4 and at the proximal end of the second distal length 3.

In accordance with an embodiment, said small bridge 8 is connected to the first proximal portion 4 and second distal portion 3 by means of welding, for example by means of a Silver-Tin welding, preferably with 95% Sn and 5% Ag.

In accordance with an embodiment, said small bridge 8 in the lengths thereof which are coupled to the first proximal portion 4 and second distal portion 3 is wound together with the body 19 by a coil wire or coil 68, for example in Platinum-Tungsten, preferably 95% Pt and 5% W.

In accordance with an embodiment, said wound wire is welded to the small bridge and body, for example by means of a Silver-Tin welding, preferably with 95% Sn and 5% Ag.

In accordance with an embodiment, said small bridge 8 is made of a shape-memory material, for example of a superelastic material, such as Nitinol.

In accordance with an embodiment, said small bridge 8 is made of stainless steel.

In accordance with an embodiment, said elongated body or core wire 19 is made of stainless steel or Nitinol.

In accordance with an embodiment, said coil wound wire or coil 68 is made of radio-opaque material, for example of a material suitable to provide a marker. Preferably, said wire is wound just before and just after the intermediate length 6 or hinge.

In accordance with an embodiment, said coil wound wire 68 has a pitch p, p′, p″, p″′ of the coils varying from a length thereof to another length thereof. For example, the coil pitch is greater at the intermediate portion 6. Alternatively, at the intermediate portion 6 said coil wound wire 68 is completely absent.

In accordance with an embodiment, said intermediate portion 6 or guide wire hinge is arranged at 2%-3% the length of the guide wire 1 from the distal end 20 of the guide wire.

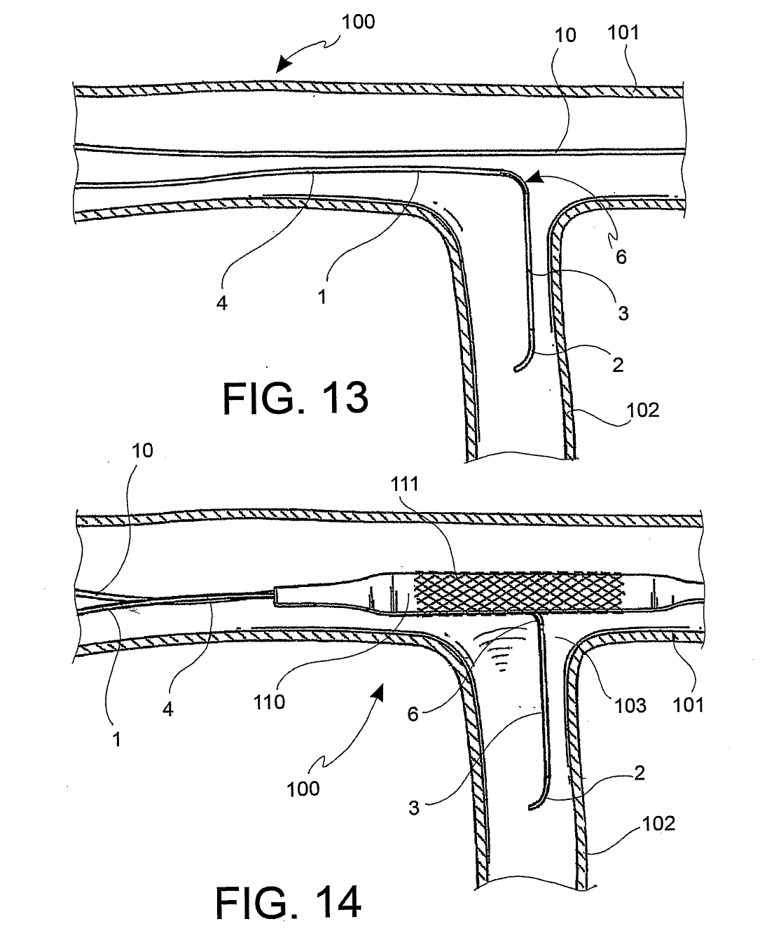

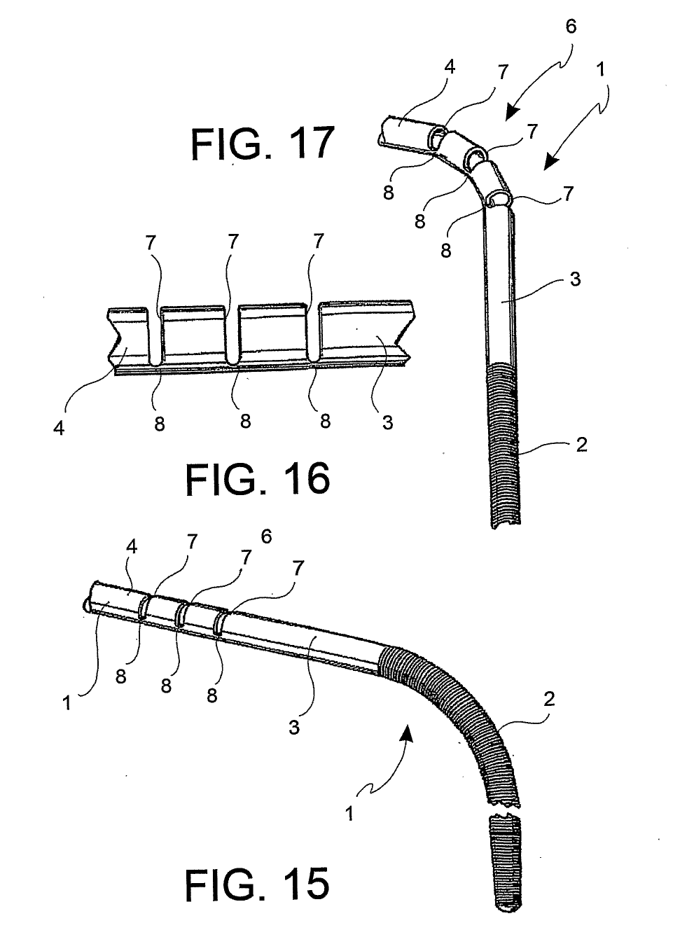

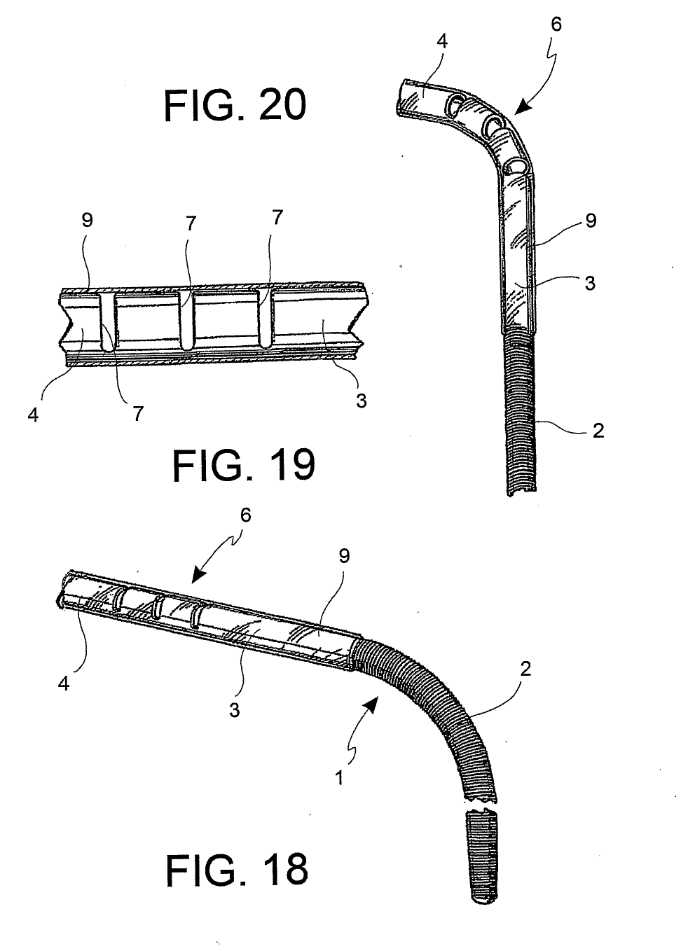

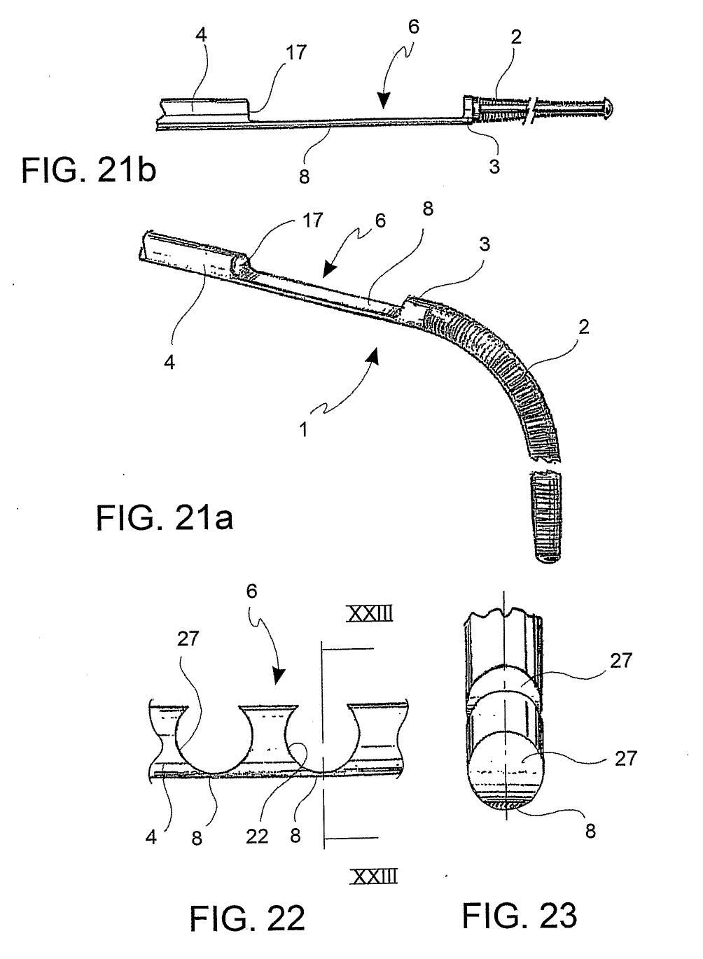

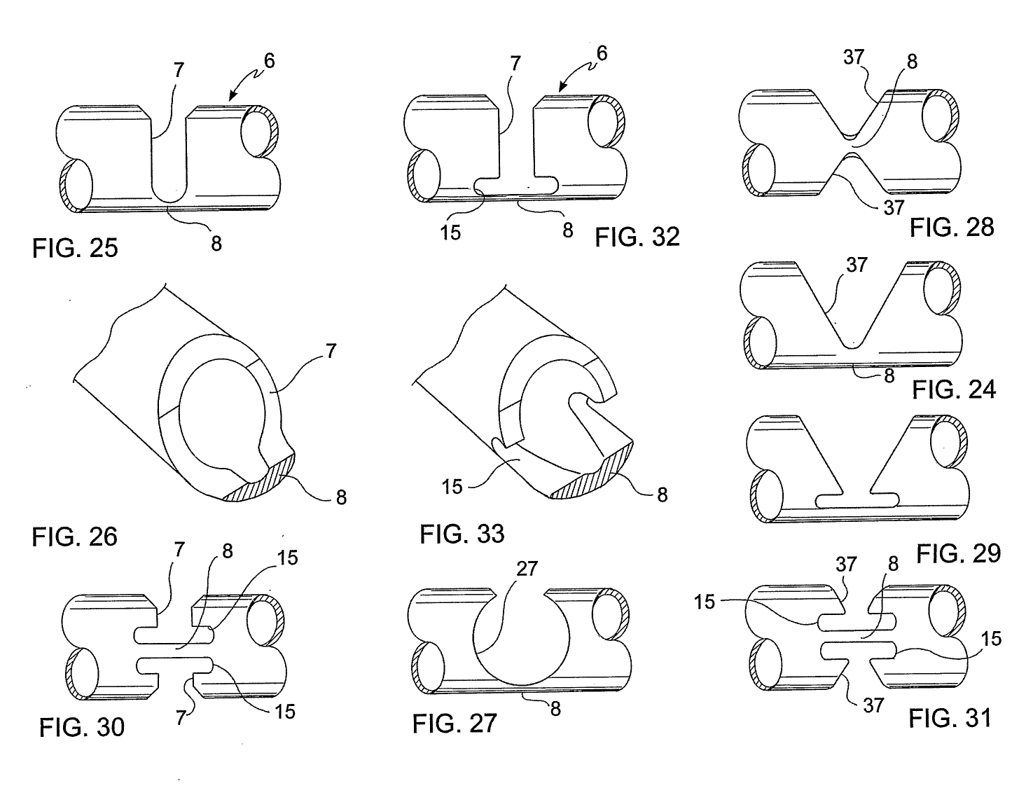

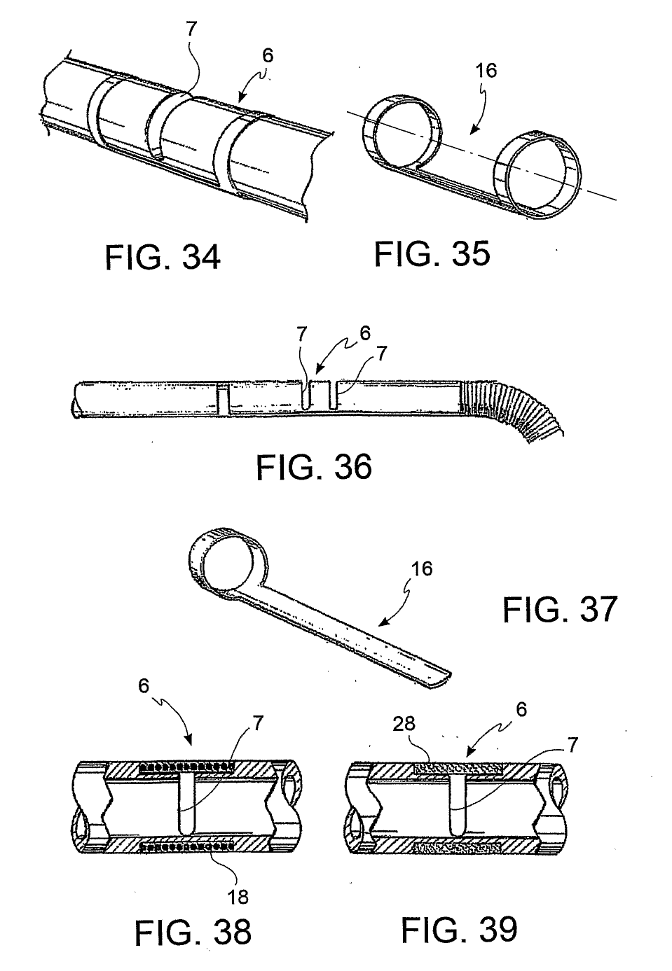

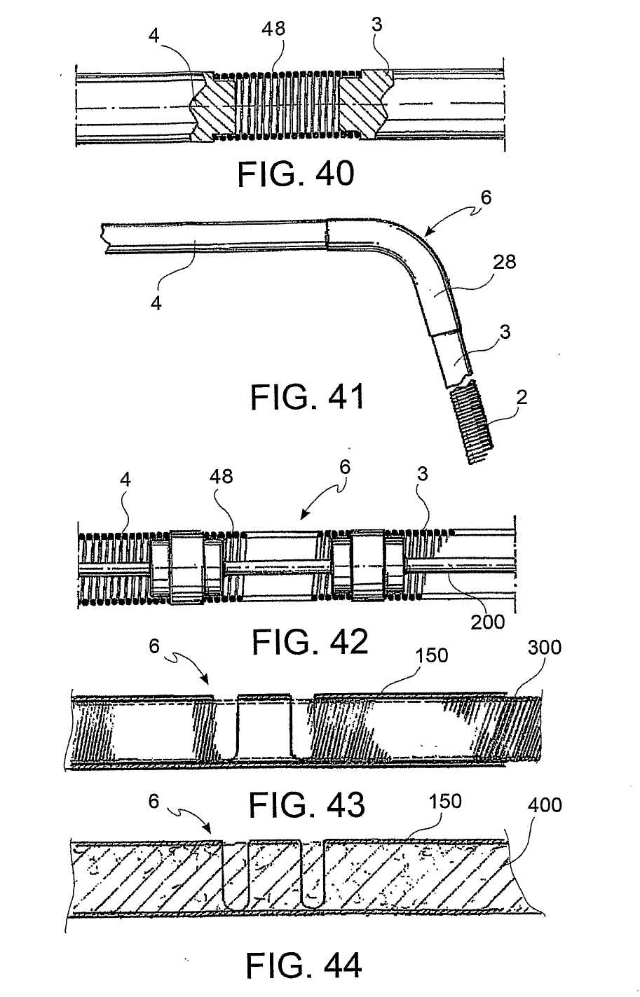

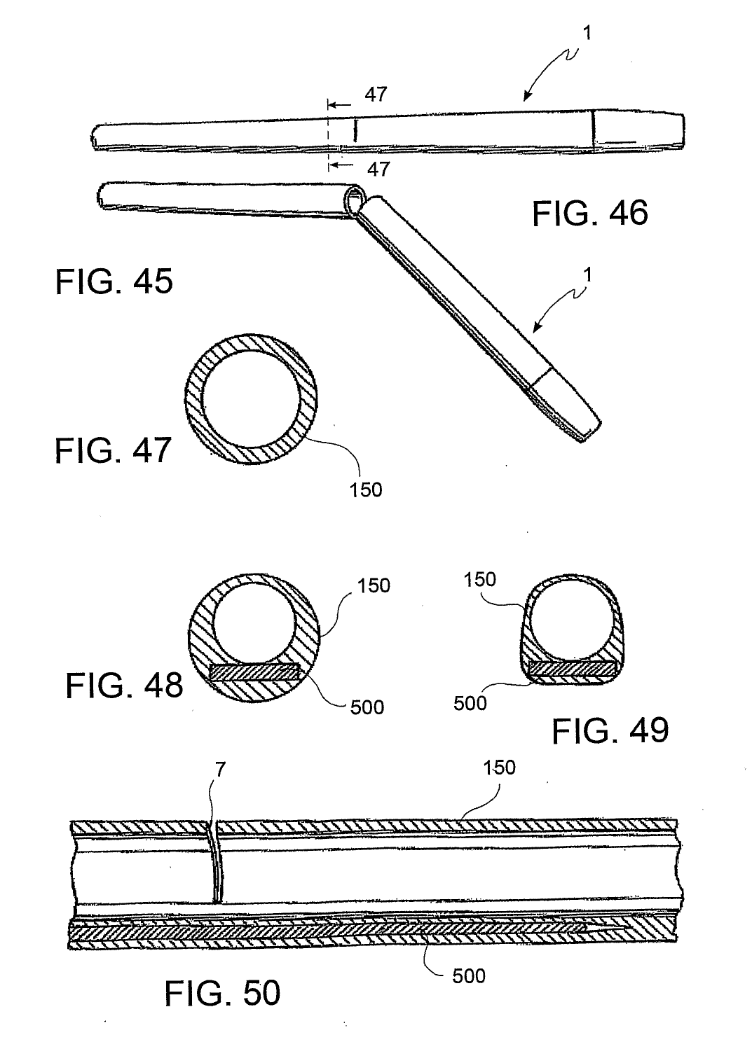

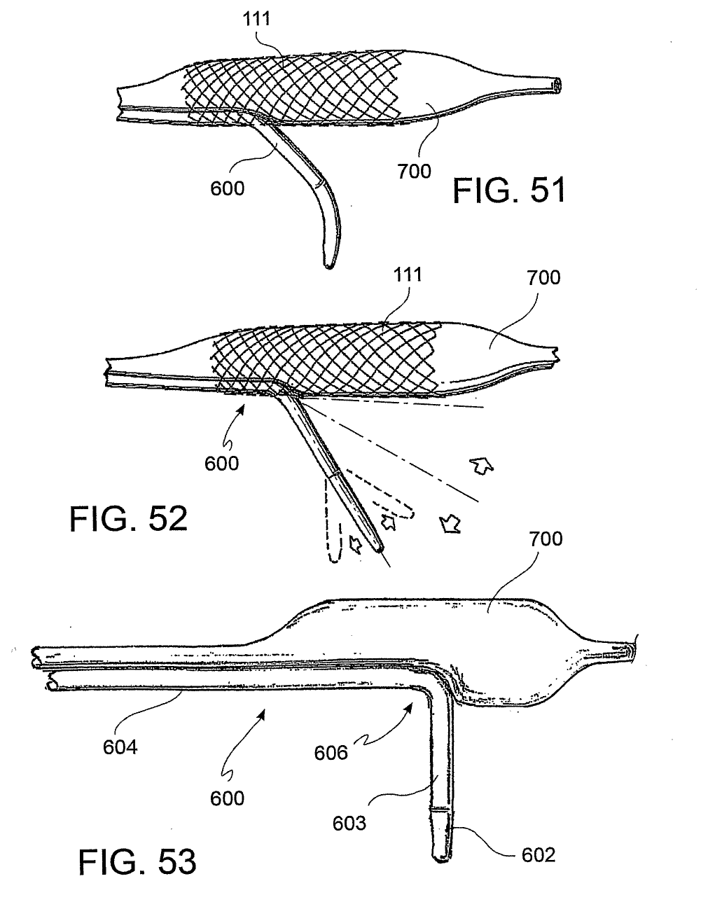

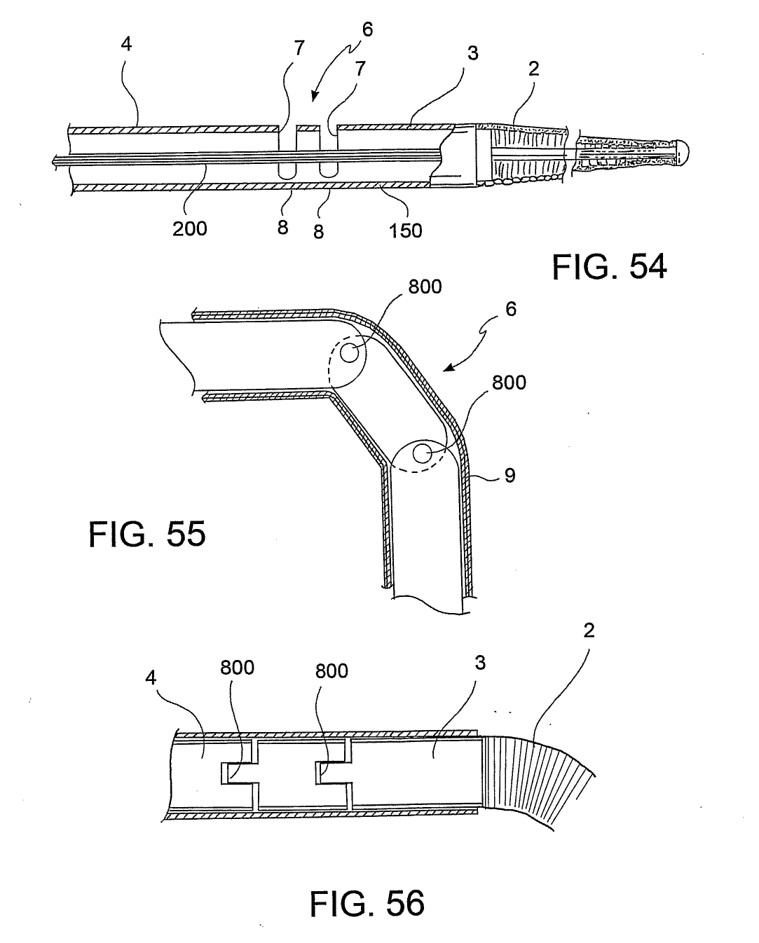

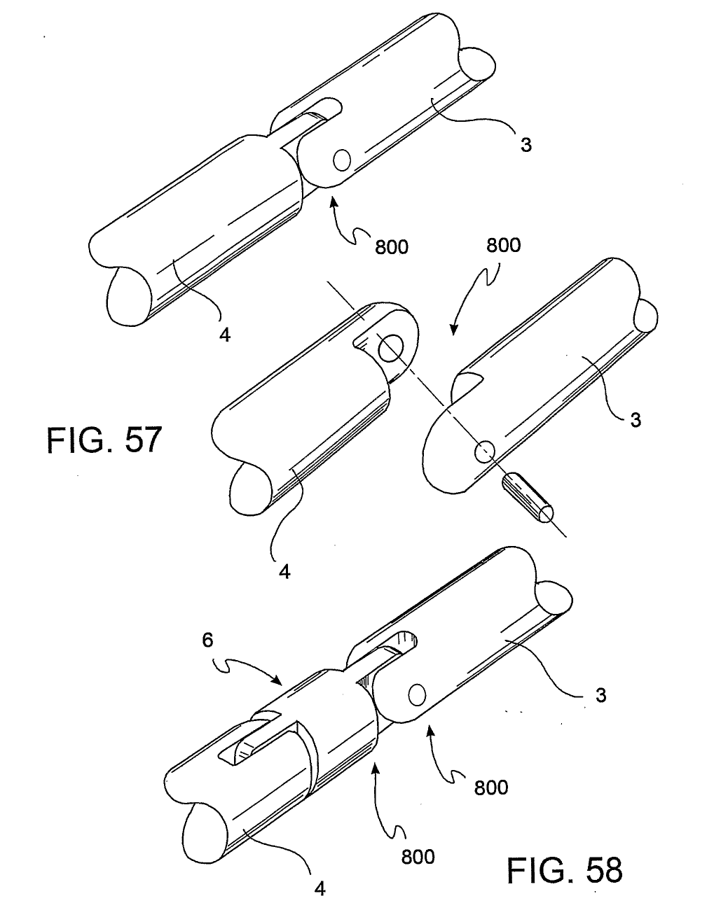

In accordance with an embodiment, said intermediate portion 6 or guide wire hinge is arranged at 2.5%-2.7% the length of the guide wire 1 from the distal end 20 of the guide wire. Preferably, this solution is used to treat the lesions in T- or Y-bifurcations. For example, said second distal portion 3, is inserted into a side branch 102 of a vessel leaving the first proximal portion 4 in the main branch 101 and the intermediate length 6 at the mouth of the side branch (FIG. 13).

In accordance with an embodiment, said intermediate portion 6 or guide wire hinge is arranged at 4.5%-6.5% the length of the guide wire 1 from the distal end 20 of the guide wire. Preferably, this solution is used to treat the ostium lesions, in which the lesion involves the mouth of a vascular duct branch. For example, a Stent Delivery System SDS 110 or catheter, for example with the baloon, is inserted into the side branch 102 or second branch of a Y-bifurcation, leaving the guide wire 1 in the main branch 101 or first branch of a Y-bifurcation, as a feedback of the proper positioning of SDS 110 perfectly inserted in the mouth of the side branch 102 with, for example an endoluminal prosthesis 111 thereof being fitted on the balloon and perfectly aligned with a proximal edge 66 thereof to the mouth or ostium of the side branch without this prosthesis 111 exiting the side branch thereby hindering the main branch lumen (FIG. 69 to 73).

In accordance with an embodiment, at said intermediate portion or hinge 6 said guide wire 1 is pre-shaped with a predetermined curvature, thereby providing a predetermined angle A between said first proximal portion 4 and said second distal portion 3.

In accordance with an embodiment, said intermediate length or hinge 6 has a body portion or core wire 19 not provided with notches and bent or kinked 67, for example to create a predetermined narrowing forming a small bridge 8 connecting said first proximal portion 4 to said second distal portion 3. Said bending 67 provides a predetermined angle A between said first proximal portion 4 and said second distal portion 3 of the guide wire. Advantageously, before and/or after said bending or kinking 67 markers can be provided. In accordance with an embodiment, before and/or after the intermediate length 6 a coil wound wire is provided, preferably of a radio-opaque material.

In accordance with an embodiment, said guide wire 1 is covered on at least one portion thereof which comprises the intermediate length or hinge 6 by a lining or jacket 63.

In accordance with an embodiment, said guide wire 1 is covered on at least one distal portion thereof by a lining or jacket 63.

In accordance with an embodiment, said guide wire 1 is covered on at least one distal portion thereof by a polyurethane lining or jacket (63).

In accordance with an embodiment, said guide wire 1 is covered on at least one portion thereof by a lining or jacket (63) covered by a hydrophilic layer.

In accordance with an embodiment, at the proximal end of the wire 21 a coupling 64, 65 is provided for an extension or doc extension guide wire.

In accordance with an embodiment, a method for providing a shaping of a guide wire according to any preceding embodiment, comprising the steps of:
providing a tool equipped with a rectilinear portion 42 and a portion 41 with a length shaped with a predetermined curvature of a predetermined curvature radius 43, forming a slit 44 therebetween, wherein at least the proximal portion 4 of the guide wire can be inserted;
the guide wire 1 is positioned such that an intermediate length 6 thereof is arranged between a first proximal length 4 thereof and a second distal length 3 thereof at the portion of the tool with portion 41 having a predetermined curvature 43,
a predetermined traction F-F is applied to the intermediate length 6,
a flexural moment M is applied to the wire such that the intermediate portion thereof is deformed by following the shaped length having a predetermined curvature 43, for example to provide a predetermined angle A between the proximal 4 and distal 3 lengths thereof.

1-38. (canceled)

39. A navigation guide wire through an anatomical apparatus, for example, a vascular system, comprising:
an elongated body having a proximal end, a distal end, and a longitudinal extension along an axis extending at least from said proximal end to said distal end;
at least one first proximal portion having a predetermined first flexural rigidity capable of a first predetermined flexure in at least one plane comprising at least one portion of said longitudinal axis in order to allow a first curvature at said proximal portion when bending stressed, while ensuring a predetermined elastic return in a rectilinear position of the elongated body proximal portion when not subjected to stresses;
at least one second distal portion having a predetermined second flexural rigidity, the amount of which is equal to or higher than said first flexural rigidity of said at least one first proximal portion, capable of a predetermined flexibility of said second distal portion in order to obtain a second curvature when bending stressed in at least one plane comprising at least one portion of said longitudinal axis, while ensuring an elastic return in a rectilinear position of said distal portion of said elongated body when not subjected to stress;
at least one intermediate length, arranged between said first proximal portion and said second distal portion, having a third flexural rigidity substantially lower than said first flexural rigidity and said second flexural rigidity, which allows a predetermined flexibility capable of a third curvature substantially more pronounced compared to said first curvature and second curvature, so as to allow a considerable direction change between said first proximal portion and said second distal portion, wherein a coil wound wire is arranged just before and just after said intermediate length and at the intermediate length said coil wound wire is completely absent.

40. The guide wire according to claim 39, wherein said third flexural rigidity of said intermediate length results to be substantially lower than said first flexural rigidity and second flexural rigidity mainly in a first predetermined plane comprising at least one portion of the longitudinal axis of said first intermediate length, said intermediate length having, in a second plane, orthogonal to said first plane, a fourth flexural rigidity substantially higher than said third flexural rigidity, which allows a predetermined reduced flexibility capable of a fourth curvature less pronounced than said third curvature in said fourth orthogonal plane.

41. The guide wire according to claim 39, wherein at least one further distal portion is provided distally to said distal portion, having a higher flexibility than said second distal portion, so as to turn out to be flexible or floppy so as to turn out to be adapted to negotiate even tortuous branches, for example, in a vascular system, and/or wherein proximally to said proximal portion at least one further proximal portion is provided, which advantageously has at least one fifth flexural rigidity substantially greater than said first flexural rigidity.

42. The guide wire according to claim 39, wherein said intermediate length comprises at least one notch transversal to said elongated body co-operating with a small bridge connecting said first proximal portion to said second distal portion and/or wherein said intermediate length comprises at least one notch transversal to said elongated body providing a partition and separating said first proximal portion from said second distal portion.

43. The guide wire according to claim 39, wherein said first proximal portion is connected to said second distal portion by means of a small bridge, and/or wherein said small bridge is a rectangular section ribbon bridge, preferably said small bridge is coupled to flattenings provided at the distal end of the first proximal length and at the proximal end of the second distal length, and/or wherein said small bridge is connected to the first proximal portion and second distal portion by means of welding, for example by means of a Silver-Tin welding, preferably with 95% Sn and 5% Ag, and/or wherein said small bridge in the lengths thereof which are coupled to the first proximal portion and second distal portion is wound together with the body by a coil wire or coil, for example in Platinum-Tungsten, preferably 95% Pt and 5% W, and/or wherein said wound wire is welded to the small bridge and body, for example by means of a Silver-Tin welding, preferably with 95% Sn and 5% Ag and/or wherein said small bridge is made of a shape memory material, for example a superelastic material and/or wherein said small bridge is made of stainless steel.

44. The guide wire according to claim 39, wherein said elongated body or core wire is made of stainless steel or Nitinol and/or wherein said coil wound wire or coil is made of a radio-opaque material to provide a marker.

45. The guide wire according to claim 39, wherein said intermediate portion or guide wire hinge is arranged at 2%-3% the length of the guide wire from the distal end of the guide wire and/or wherein said intermediate portion or guide wire hinge is arranged at 2.5%-2.7% the length of the guide wire from the distal end of the guide wire and/or wherein said intermediate portion or guide wire hinge is arranged at 4.5%-6.5% the length of the guide wire from the distal end of the guide wire.

46. The guide wire according to claim 39, wherein at said intermediate portion or hinge said guide wire is pre-shaped with a predetermined curvature, thereby providing a predetermined angle between said first proximal portion and said second distal portion.

47. The guide wire according to claim 39, wherein said intermediate length or hinge has a body portion or core wire not provided with notches and bent or kinked to create a predetermined narrowing which provides a small bridge connecting said first proximal portion to said second distal portion.

48. The guide wire according to claim 39, wherein said guide wire is covered on at least one portion thereof which comprises the intermediate length or hinge by a lining or jacket and/or wherein said guide wire is covered on at least one distal portion thereof by a lining or jacket and/or wherein said guide wire is covered on at least one portion thereof by a polyurethane lining or jacket and/or wherein said guide wire is covered on at least one portion thereof by a lining or jacket covered by a hydrophilic layer.

49. The guide wire according to claim 39, wherein at the proximal end of the wire a coupling is provided for an extension or doc extension guide wire.

50. A method for carrying out a shaping of a guide wire according to claim 39, comprising the steps of:
providing a tool provided with a rectilinear portion and a portion with a shaped length with a predetermined curvature of a predetermined curvature radius providing a slit therebetween,
positioning the guide wire such as to arrange an intermediate length thereof arranged between a first proximal length thereof and a second distal length thereof at the portion of the tool with portion with predetermined curvature,
a predetermined traction is applied to the intermediate length,
a flexural moment is applied to the wire such that the intermediate portion thereof is deformed by following the predetermined curvature shaped length.
